(19) United States
(12) Patent Application Publication
(10) Pub. No.: US 2018/0315097 A1
(43) Pub. Date: Nov. 1, 2018

(54) DYNAMIC MARKETING AND PRICING SYSTEM FOR SENIOR LIVING COMMUNITIES

(71) Applicant: SENIORVU, INC., Kansas City, KS (US)

(72) Inventors: Timothy J. DONNELLY, Leawood, KS (US); Paul T. Goldman, Augusta, KS (US); Daniel J. Cates, Overland Park, KS (US); Nicholas M. Peeples, Prairie Village, KS (US)

(73) Assignee: Seniorvu, Inc., Kansas City, KS (US)

(21) Appl. No.: 15/967,799

(22) Filed: May 1, 2018

Related U.S. Application Data
(60) Provisional application No. 62/492,885, filed on May 1, 2017.

(51) Int. Cl. G06Q 30/02; G16H 10/60

(52) U.S. Cl. CPC G06Q 30/0276; G16H 10/60; G06Q 30/0283; G06Q 30/0244; G06Q 30/0249

(57) ABSTRACT

The present invention provides systems and processes for determining optimal marketing plans and optimal pricing for senior residents of a senior living community based on the historical costs of successfully marketing to certain segments of the population of seniors in a target area, the costs associated with providing certain types of senior care, as well as the senior living community's desired or required profit margin. Various aspects of the invention may be implemented on an online platform, a public or corporate network, a private network server, a personal computer system or mobile device, such as a smart phone or tablet computer.

### TECHNICAL FIELD

The present invention relates generally to systems and methods for determining suitable prices for residents and potential residents of senior living communities, and more particularly to computer-implemented systems and processes that automatically provide senior living community operators with suitable prices for potential residents based on the marketing costs and desired profit margins of the senior living communities, as well as the attributes (personas) of the potential senior residents.

### BACKGROUND

In today's senior living community industry, there is no standard, quantitative and data driven system or method that a senior living community operator can use to determine the best marketing plan to use to successfully attract and convince a potential individual senior resident (or a collection of potential senior residents having a certain profile) to move into a senior living community. There is also no standard, quantitative and data driven system or method that a senior living community operator can use to determine and set the best price for a prospective individual senior resident or a collection of prospective senior residents. Consequently, many senior living community operators rely on guesses, hunches and luck to market and price their services, instead of hard data and analysis. This reliance leads to a significant amount of over-pricing and underpricing in the senior living community industry, which in turn leads to substantial losses in revenue and profits.

Therefore, there is considerable need in the industry for a system and method for determining a suitable price to offer to a particular senior based on the attributes of the senior living community, as well the attributes of the particular senior (such as the types of care and the services required by the senior, the senior's life expectancy, and the senior's financial condition). There is also considerable need in the industry for a system and method of determining a suitable price and a suitable marketing plan for convincing an influential senior to move into a senior living community based on the attributes of the influential senior, as well as the influential senior's network of family and friends who might be influenced by the influential senior to move into the same senior living community.

### DISCLOSURE OF THE INVENTION

The present invention addresses these needs by providing systems and processes for determining optimal marketing plans and optimal pricing for senior residents of a senior living community based on the historical costs of successfully marketing to certain segments of the population of seniors in a target area, the costs associated with providing certain types of senior care, as well as the senior living community's desired or required profit margin. Various aspects of the invention may be implemented on an online platform, a public or corporate network, a private network server, a personal computer system or mobile device, such as a smart phone or tablet computer.

Embodiments of the present invention may be configured to help a senior living community operator determine an optimal marketing plan for a potential customer having certain attributes and a certain life expectancy, as well as a suitable price that should be offered to that potential customer in view of the potential customer's life expectancy, the expected costs for marketing to that potential customer, and the expected expense that the senior living community will incur for providing care to that potential customer after the potential customer moves into the community. The system also automatically calculates how much money the operator can reasonably expect to spend on marketing efforts (i.e., a suitable marketing budget) to attract that potential customer (or persons having similar attributes to that potential customer).

Suppose, for example, that a senior living community charges residents $4,000 per month per room for a certain type of senior care, and it costs the senior living community $2,000 per month to provide that type of senior care. Suppose further that the senior living community operator has an internal goal of spending no more than 5% of expected annualized profit on marketing efforts directed to candidates requiring that type of senior care. This would mean that the operator can spend up to 1,200 (or 5%*2,000*12) per year on marketing efforts designed to attract and persuade suitable seniors to move into the $4,000 per month room. Providing these inputs to embodiments of the present invention would permit the system operator to automatically determine what form of marketing can be had for $100 per month ($1,200 per year), as well as a success rate for those forms of marketing in respect to certain types of senior candidates (i.e., seniors having a certain profile).

In one implementation, the system may be configured to automatically calculate and provide a senior living community operator with an optimal price for a room based on the type of senior care required, the cost of providing that type of senior care, the amount of money typically required to run a successful marketing campaign for that room based on previously-used marketing campaigns and the responses of previously targeted similar candidates, and the cost associated with providing that room and type of care. Success may be measured in a variety of different ways, including without limitation, whether or not a candidate moves in, takes a tour, responds favorably to a flyer or email, visits a website, calls the senior living community for more information, etc. within a certain time period after initiation of the marketing plan. The optimal price may also be based in part on the actions and reactions of particular senior candidates to certain elements (i.e., tasks) encompassed by the selected marketing plan.

In another implementation, the system may be configured to automatically calculate an optimal marketing budget for a room based on a given price for the room, a given cost associated with providing the room (and associated senior care), a given desired profit margin, and the success rates of previously executed marketing plans directed at previous candidates with the same or similar personas and/or attributes. In preferred embodiments, the system will automatically select and initiate on behalf of the senior living community an initial marketing plan directed at senior candidates having certain profiles (i.e., seniors with suitable personas), automatically track the responses (or non-responses) of those targeted senior candidates, and automatically revise and restart a marketing plan if that marketing plan does not meet a specified success metric within a specified time limit.

### BRIEF DESCRIPTION OF THE DRAWINGS

The present invention and various aspects, features and advantages thereof are explained in detail below by reference to the exemplary and therefore non-limiting embodiments shown in the figures, which constitute a part of this specification and include depictions of the exemplary embodiments. In these figures:

FIG. 1 shows a high-level block diagram of a marketing and pricing system configured to operate according to one implementation of the present invention.

FIG. 2 shows a high-level flow diagram illustrating by way of example the overall process flow for one embodiment of the invention.

FIGS. 3 and 4 together show a high-level flow diagram illustrating by way of example the steps performed to determine an optimal marketing plan and an optimal marketing budget for a seniors in a target area, based on their senior attributes.

FIG. 5 shows a high-level flow diagram illustrating by way of example the steps performed to determine an optimal price for a well-matched senior in one implementation of the invention.

FIG. 6 shows examples of community attributes in one implementation of the invention.

FIG. 7 shows examples of senior attributes in one implementation of the invention.

FIG. 8 shows examples of market conditions that could be used in one embodiment of the invention to determine an optimal marketing plan and an optimal price.

### DETAILED DESCRIPTION OF EXEMPLARY EMBODIMENTS

A marketing plan may be described as any plan, information, document, proposal or strategy designed to help a business make the best use of its resources to achieve sales and growth objectives, or increase company or product recognition and awareness. Typically, a marketing plan outlines marketing campaigns and marketing activities for a business for a set time frame. The marketing plan may also propose and describe business activities designed to accomplish specific marketing objectives within the set time frame. The marketing plan may also include a description of the current marketing position of a business, a discussion of the target market, budgets, sales forecasts, strategies, projected financial statements, and a description of the mix of marketing campaigns, marketing activities and marketing tasks that a business will use to achieve its marketing goals during the specified time frame. For example, a marketing plan may include a strategy to increase the business' market share by fifteen percent. The marketing plan would then outline the marketing campaigns, marketing activities and marketing tasks that need to be implemented by the business to reach the fifteen percent increase in the business market share. Marketing activities may include, for example, making and broadcasting radio and television commercials promoting a business' products and services, sending out emails containing newsletters or articles that promote a business' products or services, creating a website and taking steps to ensure that the new website is always among the top results on search engines (search engine marketing), placing a business' products or services on outdoor media (billboards) or in a movie or video game, or telemarketing to potential customers. Marketing tasks may include any or all the specific tasks that need to be completed to carry out a marketing activity for a marketing campaign comprising part of a marketing plan. For example, if the marketing activity is to send out emails promoting products or services, then the marketing tasks associated with such a marketing activity could include, for example, creating a new subject line for the marketing email, creating a new image for the marketing email, creating a hyperlink to embed in the marketing email, and/or creating a new web page that can be accessed by the hyperlink embedded in the marketing email.

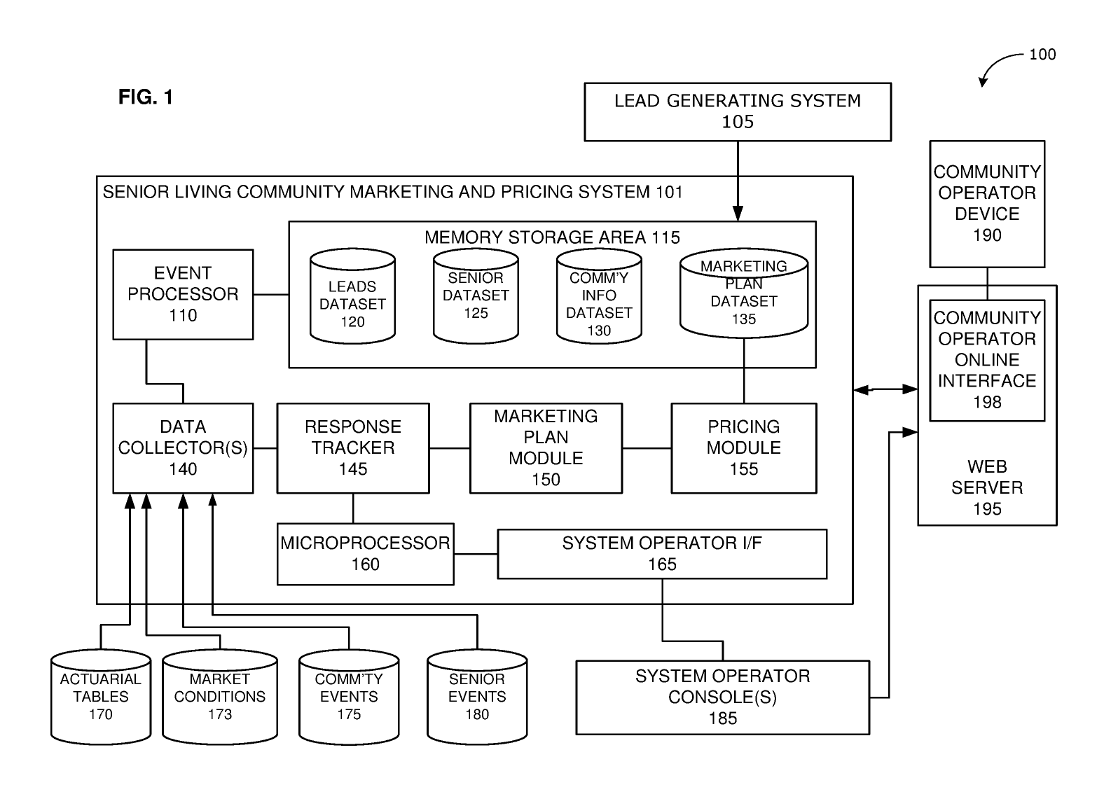

Turning now to the figures, FIG. 1 shows a high-level block diagram of a computer network 100. The computer network 100 includes a senior living community marketing and pricing system 101 configured to operate according to one implementation of the present invention. As shown in FIG. 1, the computer network 100 also includes a lead generating system 105, and several external data sources, including an actuarial tables database 170 to supply life-expectancy information, a market conditions database 173 to supply market condition information, such as interest rates, weather, population health, census data, etc. These and other non-limiting examples of market conditions information that could be used in one embodiment of the invention are listed in FIG. 9. The collection of external data sources also may include a community events database 175 to supply news and information about senior living communities in a target area, and a senior events database 180 to supply news and information about senior citizens in a target population.

In accordance with the implementation illustrated in FIG. 1, the senior living community marketing and pricing system 101 includes a microprocessor 160 and a collection of computer software programs located in executable memory (for clarity in the diagram, the executable memory is not shown in FIG. 1). The computer software programs in the executable memory contain program instructions that, when executed by the microprocessor 160, will cause the microprocessor 160 to carry out the functions of the senior living community marketing and pricing system 101 to dynamically provide senior living community operators with marketing plans and pricing information suitable for the senior living community operator to use for marketing senior living community housing and services to seniors who meet certain matching criteria (i.e., “optimal” senior candidates) The senior living community marketing and pricing system 101 further includes a memory storage area 115 (also referred to as secondary memory) containing a plurality of datasets (i.e., databases) containing structured records of information required and/or produced by the senior living community marketing and pricing system 101. The plurality of datasets includes a leads dataset 120, a senior dataset 125, a community information dataset 130 and a marketing plan dataset 135, all of which will be discussed in more detail below.

One or more data collectors 140 is provided on the senior living community marketing system 101 to monitor and collect relevant data from the external data sources 170, 173, 175 and 180. In some embodiments, the data collectors 140 comprise an interface to a data subscription service, while in other embodiments, the data collector 140 may comprise a web crawler or webpage scraper configured to identify and acquire useful market data, pricing data and competition data from data sources on the Internet. In still other embodiments, the data collectors 140 may comprise a combination of data subscription services, web crawlers and webpage scrappers. Also provided on the senior living community marketing and pricing system 101 is an event processor 110, which is configured to sort, filter, aggregate and format, if necessary, all the data collected by the data collectors 140, and then store the pertinent data in the appropriate datasets in the memory storage area 115 for use and further processing by other components of the system, as herein described below.

The senior living community marketing and pricing system 101 further includes a system operator interface 165, which permits a system operator to interact with the senior living community marketing and pricing system 101 via a connected system operator console 185. With this connection, the system operator can use the system operator console 185 to provide operating parameters and tune configuration settings on the system, and receive and view the information and data produced by the senior living community marketing and pricing system 101. The system operator may also be a person having special knowledge and experience about how the senior living community industry operates under certain circumstances, about what the outputs typically mean in practice, or how the senior living community industry works, and may therefore sometimes play a role as an expert consultant or advisor to the senior living community using the system to develop marketing plans and budgets and pricing.

The computer network 100 also includes one or more connections to a web server 195. The web server 195 typically supports a community operator online interface 198, which permits a community operator using a community operator device 190, such as a desktop computer, laptop computer, tablet computer or smartphone, to use the Internet and an Internet browser program to access and provide operating parameters (such as search criteria) and other information (such as senior living community attributes, senior living community amenities and budgets) to the senior living community marketing and pricing system 101, and receive and view the system outputs, such as marketing plans, pricing plans, and reports, designed to help the operator of the senior living community optimize marketing campaigns and marketing activities designed to persuade an optimal senior candidate make a decision to move into the senior living community.

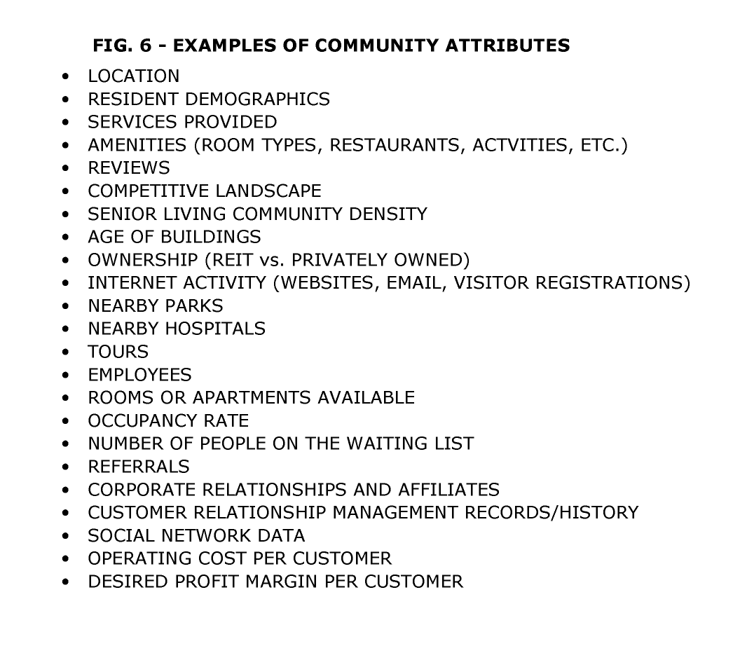

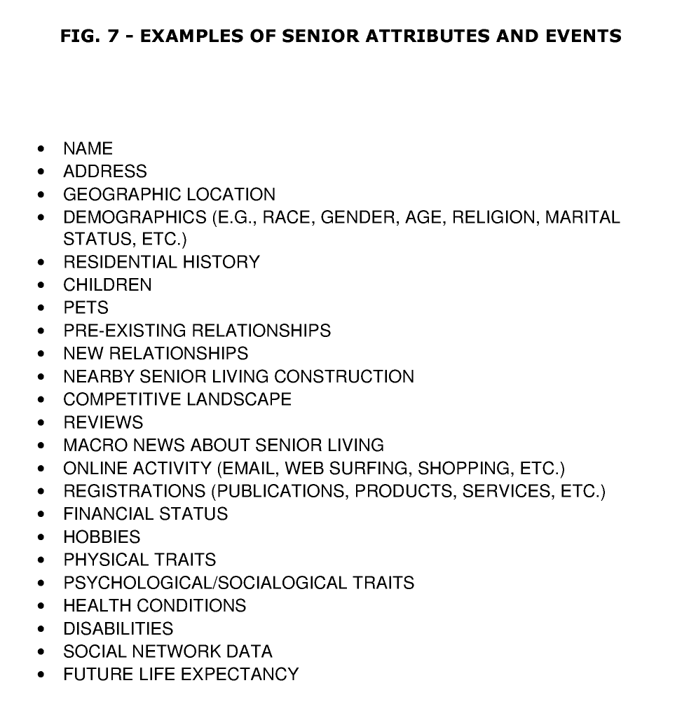

Co-pending U.S. application Ser. No. 15/485,129 and PCT Application No. PCT/US2017/027055, both of which are incorporated in their entirety by this reference, describe in detail the features and operation of a lead generation system 105 that could be used in connection with embodiments of the present invention. In general, the lead generating system 105 identifies and supplies information to the senior living community marketing and pricing system 101 about eligible seniors who have been identified by the lead generation system as a good “match” with the senior living community based on the attributes of the senior and the attributes of the senior living community. Examples of community attributes are shown in FIG. 6, while examples of senior attributes are shown in FIG. 7. Preferably, the lead generation system is connected to a variety of public subscription-based data services so that it can acquire and use early indicator data from an early indicator data source (such as a database of home sales); to identify seniors who are likely to need housing and senior care in the near future. To this end, an event processor on the lead generation system 105 is configured to process the early indicator data to detect an early indicator for the senior care type, a potential customer for the senior care type, customer demographic attributes for the potential customer, and senior events associated with the potential customer.

The lead generating system 105 finds optimal senior candidates for a senior living community based on demographic and event qualifiers, demographic attributes of the current population of the senior living community, and weights assigned to the demographic qualifiers, trait qualifiers and event qualifiers. The weights are provided to the senior living community marketing and pricing system 101 by the senior living community operator (or by a system operator) via the web server 195 and community operator online interface 198. All of this information about an optimal senior is transmitted to the senior living community marketing and pricing system 101 of the present invention and stored in the senior dataset 125 for use by other components of the system 101 for the purpose of producing marketing and pricing data for the senior living community, and transmitting the marketing and pricing data to the community operating device 190 for access and review by the operator of the senior living community.

The lead generating system 105 may also include a persona score calculator (also not shown in FIG. 1), which creates senior personas for a target collection of potential seniors and calculates a senior persona score for the potential customer. A senior persona for a senior is a collection of attributes and attribute values associated with the senior. For example, senior citizen John Doe's senior persona may comprise multiple attributes and values for those attributes, such as: gender=“male,” religion=“catholic,” marital status=“married,” income=“$68,000,” military veteran=“no,” and college graduate=“yes.” Generating the senior persona typically comprises retrieving multiple attributes for the selected senior and the values of the attributes from the senior dataset and placing those values in a temporary array or other memory structure in preparation to run those attributes and values through the persona scoring algorithm for the community.

A senior persona score is a relative measure of how well the personal attributes of a potential customer for the senior living community matches the community attributes associated with the senior living community. Community attributes for a senior living community may include, for example, the community's location, resident demographics, services provided, amenities, occupancy rate, operating costs, desired profit margin per customer, etc. These and other examples of community attributes associated with a senior living community are shown in FIG. 6. The persona score calculator produces persona scores based on a combination of a demographic qualifier score, a trait qualifier score, an event qualifier score, or all of them, and then transmits the persona score for the potential customer to the leads dataset 120 for further processing by the senior living community marketing and pricing system 101 of the present invention.

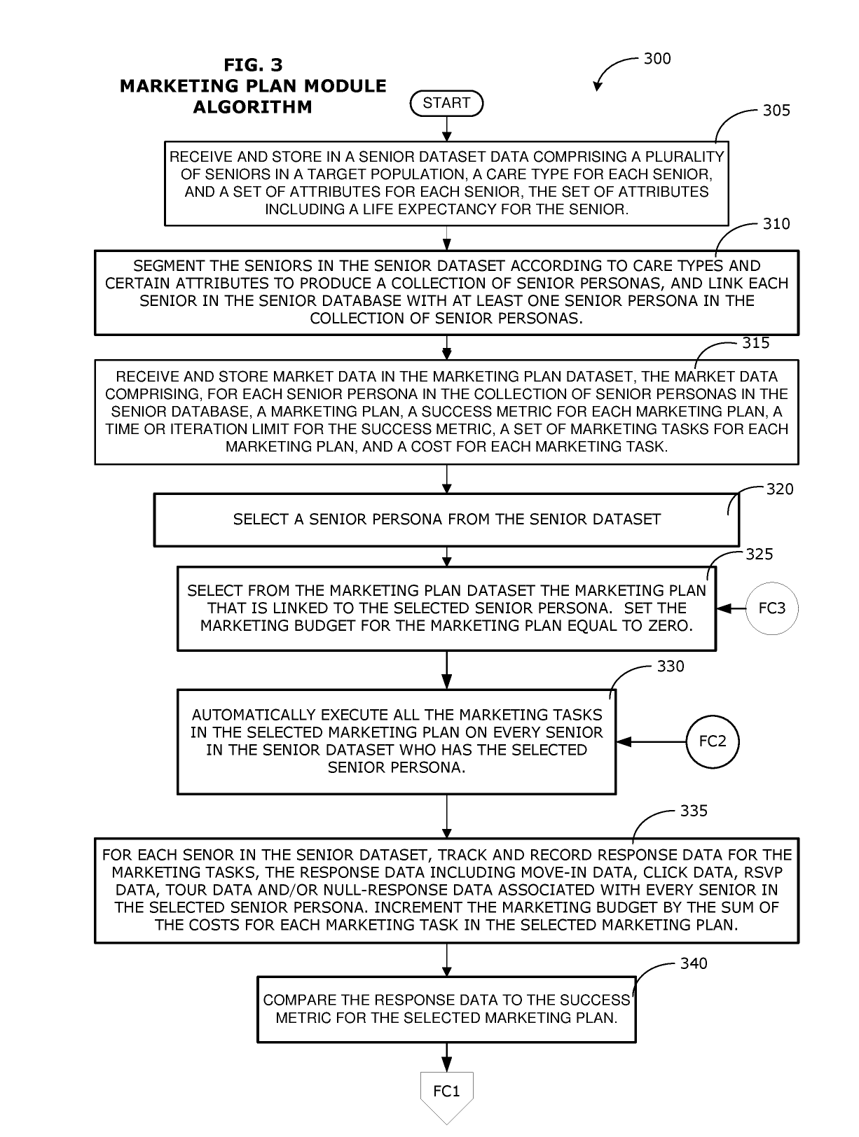

Finally, the senior living community marketing and pricing system 101 also includes a response tracker 145, a marketing plan module 150 and a pricing module 155, all of which contain program instructions that are operable with the microprocessor 160 to cause the microprocessor 160 to carry out the functions of producing an optimal marketing plan and an optimal price for an optimal senior. The algorithms carried out by the microprocessor 160 operating under the control of the program instructions in the response tracker 145, the marketing plan module 150 and the pricing module 155 are shown in FIGS. 3, 4 and 5, and explained in greater detail below.

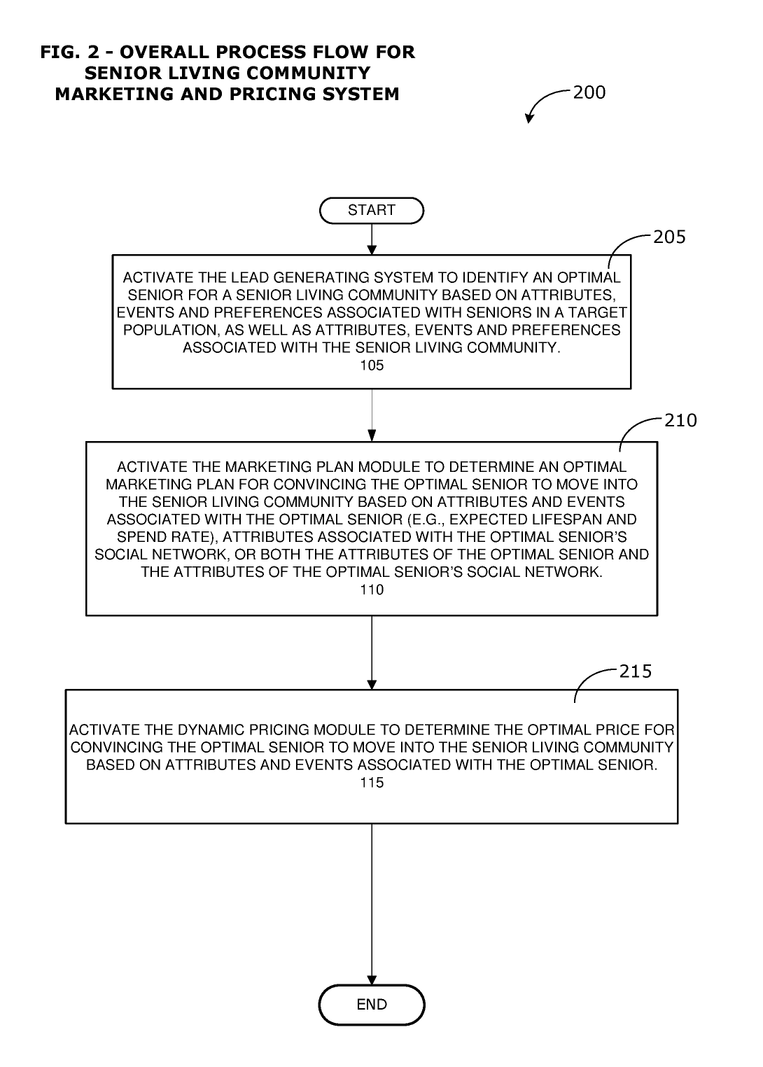

FIG. 2 shows a high-level flow diagram illustrating by way of example the overall process flow for one embodiment of the invention. As shown in step 205 of FIG. 2, the first step in the overall process flow is to activate the lead generating system 105 to identify an optimal senior for the senior living community based on attributes, events and preferences associate with seniors in a target population, as well as attributes, events and preferences associated with the senior living community. Examples of senior living community attributes are shown in FIG. 6, and examples of senior attributes and senior events associated with a senior are shown in FIG. 7. The lead generation system 105 may be incorporated into the senior living community marketing and pricing system 101, or otherwise accessed via a data communications channel as shown in FIG. 1.

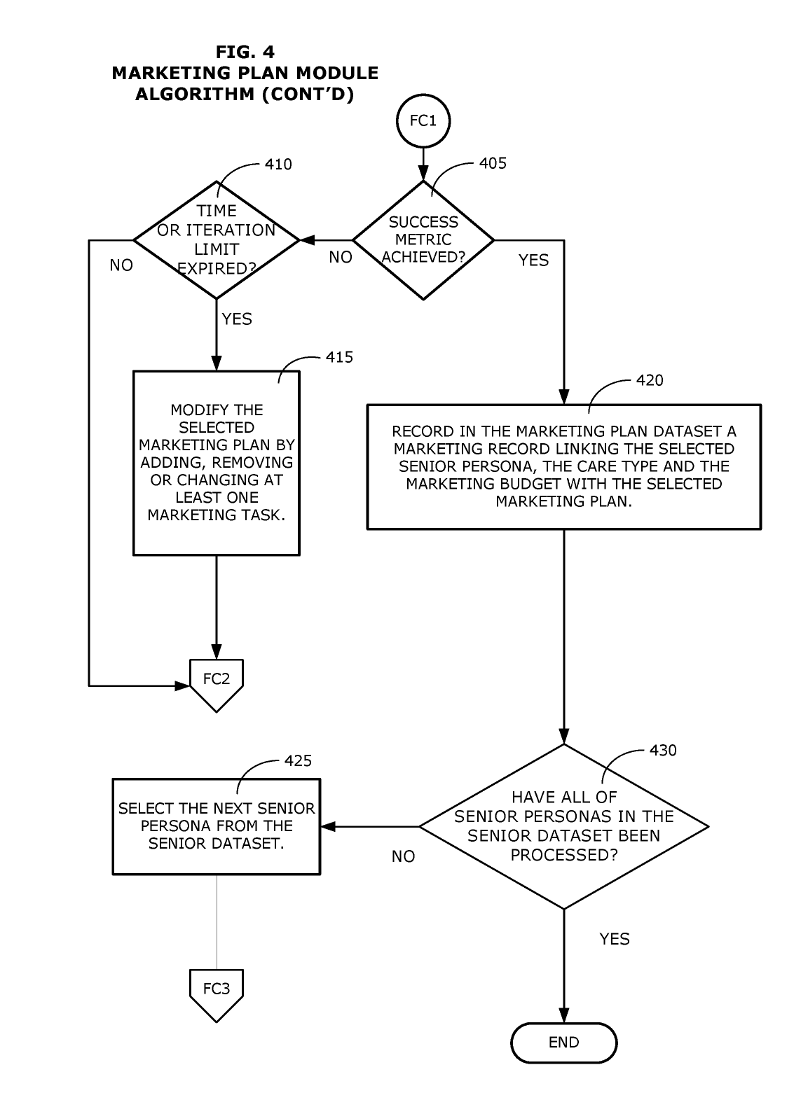

The second step in the overall process flow, step 210 of FIG. 2, is to activate the marketing plan module (shown as marketing plan module 150 in FIG. 1) to determine an optimal marketing plan to execute to try to persuade the optimal senior to move into the senior living community. The specifics of the marketing plan may be based on attributes and events associated with the optimal senior (e.g., expected lifespan and spend rate), attributes associated with the optimal senior's social network, or both the attributes of the optimal senior and the attributes of the optimal senior's social network. A high-level flow diagram illustrating the steps of the algorithm followed by the programming instructions of the marketing plan module 150 to cause the microprocessor 160 to produce the optimal marketing plan for the optimal senior is shown in FIGS. 3 and 4, which are described in greater detail below.

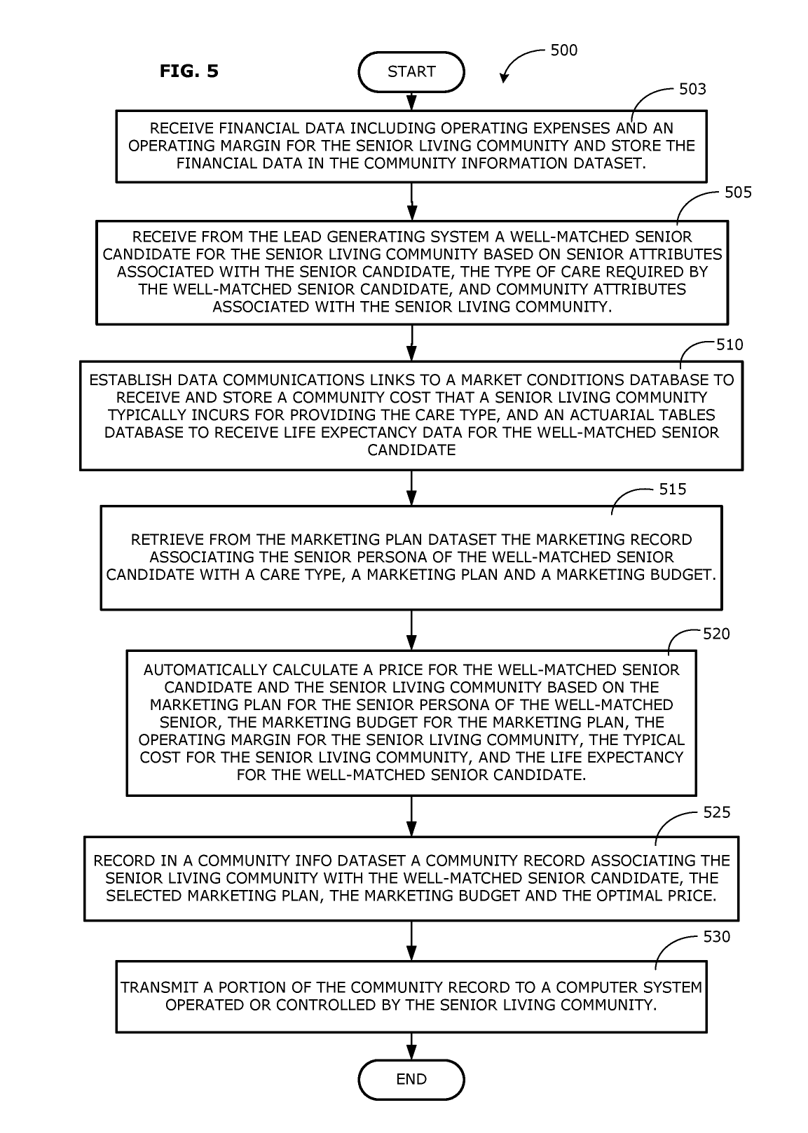

The third step in the overall process flow, shown as step 215 in FIG. 2, is to activate the dynamic pricing module (shown as pricing module 155 in FIG. 1) to determine the optimal price for the optimal senior. The optimal price comprises a price that is more likely to persuade the optimal senior to move into the senior living community based on attributes and events associated with the optimal senior. A high-level flow diagram illustrating the steps of the algorithm followed by the programming instructions of the pricing module 155 to cause the microprocessor 160 to produce the optimal price for the optimal senior is shown in FIG. 5, which is also described in greater detail below.

FIGS. 3 and 4 together show a high-level flow diagram illustrating by way of example the steps performed by the microprocessor 160 operating under the control of the response tracker 145 and marketing plan module 150 (both shown in the block diagram of FIG. 1), to produce and transmit to a user an optimal marketing plan for an optimal senior. As shown in FIG. 3, the process begins at step 305, in which the system receives and stores data in the senior dataset 125, the data comprising a plurality of seniors in a target population, a care type for each senior, and a set of attributes for each senior, the set of attributes including a life expectancy for each senior. These data are typically acquired from a combination of data subscription services, website scrapers, web crawlers and data communications links between the senior living community marketing and pricing system 101 and public data sources, such as the actuarial tables database 170 and the senior events database 180 shown in FIG. 1. Next, at step 310, the microprocessor 160, operating under the control of the marketing plan module 150, segments the seniors in the senior dataset 125 according to different care types and certain attributes to produce a collection of senior personas. The system also establishes a link between each senior in the senior dataset 125 and at least one senior persona in the collection of senior personas.

Next, in step 315, the system receives and stores data in the marketing plan dataset 135 of the memory storage area 115, the data comprising, for each senior persona in the collection of senior personas in the senior dataset 125, a marketing plan, a success metric for each marketing plan, a time or iteration limit for the success metric, a set of marketing tasks for each marketing plan, and a cost for each marketing task. Then, operating under the control of the marketing plan module 150, the microprocessor 160 selects one of the senior personas from the senior dataset 125 (step 320), and also selects from the marketing plan dataset 135 the marketing plan that is linked to the selected senior persona (step 325). The microprocessor also sets the marketing budget for the marketing plan equal to zero. At this point, the marketing plan module 150 causes the microprocessor 160 to automatically execute all the marketing tasks for the selected marketing plan on every senior in the senior dataset 125 who has the senior persona selected from the senior dataset 125. See step 330. While the marketing tasks in the selected marketing plan are being executed, the microprocessor 160, operating under the control of the response tracker 145, tracks and records response data for the executed marketing tasks, the response data including, for example, move-in data, click data, RSVP data, tour data and/or null-response data associated with every senior in the selected senior persona. The microprocessor 160 also increments the marketing budget by the sum of the costs for each marketing task in the selected marketing plan. See step 335.

The step of tracking and recording response data for the executed marketing tasks may further include tracking and recording, for each senior in the senior dataset, a tally of positive responses received in response to the marketing task, a tally of negative responses received in response to the marketing task, a tally of non-responses for the marketing task, a tally of mouse clicks on an element of the marketing task, a tally of customer inquiries received in response to the marketing task, a tally of website visits attributable to the marketing task, a tally of registration forms received from potential customers in response to the marketing task, a tally of physical visits by potential customers attributable to the marketing task, a tally of product or service orders received in response to the marketing task, a tally of requests to be added to a distribution list in response to the each version of the defined marketing task, a tally of requests to be removed from a distribution list in response to the marketing task, a tally of social media platform likes received in response to the marketing task, a tally of email messages received in response to the marketing task, a tally of telephone calls received in response to the marketing task, or any combination of two or more of the above-listed tallies.

In step 340, the microprocessor 160, operating under the control of the programming instructions in the marketing planning module 150, compares the response data to the success metric for the selected marketing plan. Processing then continues at step 405 of FIG. 4 by way of flow chart connector FC1, in which the system checks to determine whether the success metric has been met. If it is determined in step 405 of FIG. 4 that the success metric has not been met, the system next determines, at step 410, whether a time or iteration limit has expired. If the answer is “YES” (meaning that a time or interaction limit has been met), then, in step 415, the system modifies the selected marketing plan by adding, removing or changing at least one task in the marketing plan, and then passing control back to step 330 of FIG. 3 by way of flow chart connector FC2, which is the step that calls for automatically executing the market the tasks in the market plan on every senior in the senior dataset 125. On the other hand, if it is determined at step 410 that the time or iteration limit has not expired, then step 415 that the time or iteration limit has expired, then step 415 is skipped, and control again passes to step 330 of FIG. 3 by way of flow chart connector FC2, which is the step that calls for automatically executing the marketing tasks in the marketing plan on every senior in the senior dataset 125.

If it is determined at step 405 that the success metric has been achieved, then the system next carries out step 420 in FIG. 4, which is to record in the marketing plan dataset 135 a marketing record linking the selected senior persona, the care type and the marketing budget with the selected marketing plan. At step 430, the system determines whether or not all the senior personas in the senior dataset 125 have been processed. If the answer is “YES,” then the process stops. However, if the answer is “NO,” then the system selects the next senior persona from the senior dataset 125 before passing control back to step 325 of FIG. 3 by way of flow chart connector FC3, wherein the system again selects from the marketing plan dataset 135 the marketing plan that is linked to the selected senior persona. Thus, the system will repetitively select a senior persona and a marketing plan, and execute the tasks in the market plan on every senior in the senior dataset 125 with the same senior persona until a time or iteration limit is reached.

FIG. 5 shows a high-level flow diagram 500 illustrating by way of example the steps performed by the microprocessor, operating under the control of the programming instructions in the pricing module 155 shown in FIG. 1, to determine and provide to the senior living community operator with an optimal price for a well-matched senior in one implementation of the invention. First, as shown in step 503 of FIG. 5, the marketing and pricing system 101 receives financial data including operating expenses and operating margins for the senior living community. Typically, the financial data is received from the operator of the senior living community via the community operator online interface 198 and the web server 195 shown in FIG. 1. However, the financial data could also be acquired and supplied to the marketing and pricing system 101 by a system operator working on behalf of the senior living community to set up and configure an account for the senior living community. The financial data may be stored in the community information dataset 130, but might also be stored in another dataset (not shown in FIG. 1) incorporated by or communicatively connected to the senior living community marketing and pricing system 101.

Next, at step 505 of FIG. 5, the senior living community marketing and pricing system 101 receives from the lead generating system 105 a well-matched (or optimal) senior candidate for the senior living community based on senior attributes associated with the well-matched senior, the type of care required by the well-matched senior, and a set of community attributes associated with the senior living community, which have been previously stored in the community information dataset 130 by the operator of the senior living community or the system operator. See FIG. 6 for examples of community attributes for senior living communities, and FIG. 7 for examples of senior attributes and events for seniors.

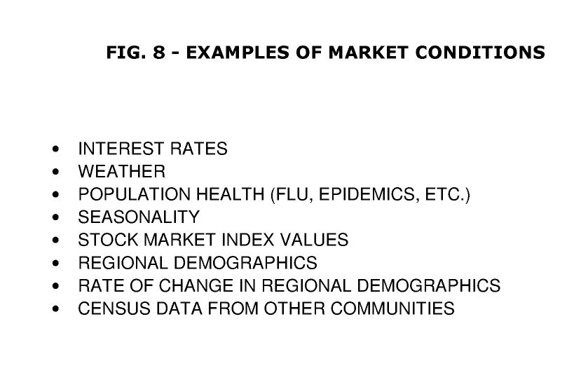

In step 510 of FIG. 5, the senior living community marketing and pricing system 101 establishes a data communications link to one or more market conditions databases, such as the market conditions database 173 shown in FIG. 1, to receive and store in the memory storage area a typical (or average) cost that a senior living community incurs for providing the type of care required by the well-matched senior. FIG. 8 shows examples of the kind of information typically stored in and provided by a market conditions database that might be used in implementations of the present invention. The senior living community marketing and pricing system 101 also establishes one or more data communications links to one or more databases of actuarial tables, such as the actuarial tables database 170 of FIG. 1, to receive and store life expectancy data for the well-matched senior candidate.

At step 515, the marketing and pricing system 101 retrieves from the marketing plan dataset 135 the marketing record associating the senior persona of the well-matched senior candidate with a care type, a marketing plan and a marketing budget. The marketing budget for the marketing plan was created by operation of the marketing plan module to carry out the steps of the algorithm shown in FIGS. 3 and 4, which was described in detail above. Then, in step 520, the microprocessor 160, operating under the control of the program instructions in the pricing module 155, automatically calculates an optimal price for the well-matched senior candidate and the senior living community based on the marketing plan for the senior persona of the well-matched senior candidate, the marketing budget for the marketing plan, the operating margin for the senior living community, the typical cost received from the market conditions database 173 for providing the type of care required by the well-matched senior candidate, and the life expectancy data received from the actuarial tables database 170 for the well-matched senior candidate.

The microprocessor then records in the community information dataset of the memory storage area 115 a record associating the senior living community with the well-matched senior candidate, the selected marketing plan, the marketing budget for the selected marketing plan and the calculated optimal price for the well-matched senior candidate. See step 525 of FIG. 5. And finally, in step 530, the senior living community marketing and pricing system 101 transmits to the community operating device 190 controlled by the operator of the senior living community at least the portions of the record comprising identification information about the well-matched senior candidate, as well as the marketing plan, the marketing budget and the optimal price for well-matched senior candidate. The record is sent to the senior living community operator by way of the community operator online interface 198 and the web server 195, whereby the community operator can review, display and/or print the record for use in executing the marketing plan with the goal of persuading the well-matched senior candidate to move into the senior living community.

Although the exemplary embodiments, uses and advantages of the invention have been disclosed above with a certain degree of particularity, it will be apparent to those skilled in the art upon consideration of this specification and practice of the invention as disclosed herein that alterations and modifications can be made without departing from the spirit or the scope of the invention, which are intended to be limited only by the following claims and equivalents thereof.

What is claimed is:

1. A marketing and pricing system for a senior living community, comprising:
a) a microprocessor;
b) a memory;
c) a first data communications link to a computing device controlled by a senior living community operator;
d) a senior dataset, in the memory, comprising a plurality of seniors in a target population, a care type for each senior, and a set of senior attributes for each senior;
e) a response tracker program, in the memory, having program instructions that, when executed by the microprocessor, will cause the microprocessor to track and record response data for a marketing activity; and
f) a marketing plan module, in the memory, that is operable with the response tracker program to cause the microprocessor to automatically:
(i) create and store in the memory a collection of senior personas by segmenting the seniors in the senior dataset according to care types and senior attributes;
(ii) link each senior in the plurality of seniors in the senior dataset with at least one senior persona in the collection of senior personas;
(iii) store in the memory a marketing plan dataset, the marketing plan dataset comprising, for each senior persona in the collection of senior personas in the senior database, a marketing plan, a success metric for each marketing plan, a time or iteration limit for the success metric, a set of marketing tasks for each marketing plan, and a cost for each marketing task in said set of marketing tasks;
(iv) select a senior persona from the senior dataset;
(v) select from the marketing plan dataset the marketing plan that is linked to the selected senior persona;
(vi) execute all the tasks in the set of tasks in the selected marketing plan on every senior in the senior dataset who has the selected senior persona;
(vii) track and record response data for the executed tasks;
(viii) increment the marketing budget by the sum of the costs for each task in the selected marketing plan;
(ix) compare the response data to the success metric for the selected marketing plan;
(x) if the response data matches the success metric, record in the marketing plan dataset a marketing record linking the selected senior persona, the care type and the marketing budget with the selected marketing plan;
(xi) if the response data does not match the success metric, modify the selected marketing plan by adding, removing or changing at least one task, and repeating steps (f)(v) through (f)(1) above until the time or iteration limit expires or the success metric is met; and
(xii) transmit at least a portion of the marketing record to the computing device controlled by the senior living community operator via the first data communications link.

2. The system of claim 1, wherein the program instructions of the response tracker are arranged to cause the microprocessor to track and record, for each senior in the senior dataset:
a) a tally of positive responses received in response to said marketing task, or
b) a tally of negative responses received in response to said marketing task, or
c) a tally of non-responses for said marketing task, or
d) a tally of mouse clicks on an element of said marketing task, or
e) a tally of customer inquiries received in response to said marketing task, or
f) a tally of website visits attributable to said marketing task, or
g) a tally of registration forms received from potential customers in response to said marketing task, or
h) a tally of physical visits by potential customers attributable to said marketing task, or
i) a tally of product or service orders received in response to said marketing task, or j) a tally of requests to be added to a distribution list in response to said each version of the defined marketing task, or
k) a tally of requests to be removed from a distribution list in response to said marketing task, or
l) a tally of social media platform likes received in response to said marketing task, or
m) a tally of email messages received in response to said marketing task, or
n) a tally of telephone calls received in response to said marketing task, or
o) any combination of two or more of the tallies enumerated in this claim.

3. The system of claim 1, further comprising:
a) a lead generating system that identifies a well-matched senior candidate for the senior living community based on senior attributes associated with the senior candidate and community attributes associated with the senior living community;
b) a second data communications link to a marketing conditions database to receive a community cost that the senior living community expects to incur for providing the care type and a community margin for the senior living community;
c) a third data communication link to an actuarial data source to receive a life expectancy for said each senior; and
d) a price module, in the memory, having program instructions that, when executed by the microprocessor, will cause the microprocessor to
(i) retrieve from the marketing plan dataset the marketing record linking the senior persona of the well-matched senior candidate with a care type, a marketing plan and a marketing budget,
(ii) calculate the price for the well-matched senior candidate and the senior living community based on the marketing plan for the senior persona of the well-matched senior, the marketing budget for the marketing plan, the community margin for the senior living community, the community cost for the senior living community, and the life expectancy for the well-matched senior candidate,
(iii) record in a community info dataset a community record associating the senior living community with the well-matched senior candidate, the selected marketing plan, the marketing budget and the optimal price, and
(iv) transmit at least a portion of the community record to the computing device controlled by the senior living community operator via the first data communications link.

4. The system of claim 1, further comprising a data collector program, in the memory, having program instructions that, when executed by the microprocessor, will cause the microprocessor to automatically acquire updated market condition data from market conditions database on a periodic basis.

5. The system of claim 4, further comprising an event processor program, in the memory, having program instructions that, when executed by the microprocessor, will cause the microprocessor to automatically filter and reformat the market condition data before storing the market condition data in the memory.

6. A method of calculating and supplying a marketing budget for a senior living community using a microprocessor and a memory, the method comprising:
a) establishing a first data communications link to a computing device controlled by a senior living community operator;
b) storing in the memory a senior dataset comprising a plurality of seniors in a target population, a care type for each senior, and a set of senior attributes for each senior;
c) with the microprocessor, producing a collection of senior personas by segmenting the seniors in the senior dataset according to care types and senior attributes;
d) with the microprocessor, linking each senior in the plurality of seniors in the senior dataset with at least one senior persona in the collection of senior personas;
e) storing in the memory a marketing plan dataset, the marketing plan dataset comprising, for each senior persona in the collection of senior personas in the senior database, a marketing plan, a success metric for each marketing plan, a time or iteration limit for the success metric, a set of marketing tasks for each marketing plan, and a cost for each marketing task in said set of marketing tasks;
f) selecting a senior persona from the senior dataset;
g) selecting from the marketing plan dataset the marketing plan that is linked to the selected senior persona;
h) executing a marketing task associated with the selected marketing plan on every senior in the senior dataset who has the selected senior persona;
i) tracking and recording response data for the marketing task;
j) incrementing the marketing budget by the sum of the costs for each task in the selected marketing plan;
k) comparing the response data to the success metric for the selected marketing plan;
l) if the response data matches the success metric, recording in the marketing plan dataset a marketing record linking the selected senior persona, the care type and the marketing budget with the selected marketing plan;
m) if the response data does not match the success metric, modifying the selected marketing plan by adding, removing or changing at least one task, and repeating steps g) through l) above until the time or iteration limit expires or the success metric is met; and
n) transmitting at least a portion of the marketing record to the computing device controlled by the senior living community operator via the first data communications link.

7. The method of claim 6, wherein the step of tracking and recording the response data for the executed marketing task comprises tracking and recording, for each senior in the senior dataset:
a) a tally of positive responses received in response to said marketing task, or
b) a tally of negative responses received in response to said marketing task, or
c) a tally of non-responses for said marketing task, or
d) a tally of mouse clicks on an element of said marketing task, or
e) a tally of customer inquiries received in response to said marketing task, or
f) a tally of website visits attributable to said marketing task, or
g) a tally of registration forms received from potential customers in response to said marketing task, or
h) a tally of physical visits by potential customers attributable to said marketing task, or
i) a tally of product or service orders received in response to said marketing task, or
j) a tally of requests to be added to a distribution list in response to said each version of the defined marketing task, or
k) a tally of requests to be removed from a distribution list in response to said marketing task, or
l) a tally of social media platform likes received in response to said marketing task, or
m) a tally of email messages received in response to said marketing task, or
n) a tally of telephone calls received in response to said marketing task, or
o) any combination of two or more of the tallies enumerated in this claim.

8. The method of claim 6, further comprising determining a price for a customer of a senior living community by:
a) establishing a second data communications link to a marketing conditions database to receive a community cost that the senior living community expects to incur for providing the care type and a community margin for the senior living community;
b) establishing a third data communication link to an actuarial data source to receive a life expectancy for said each senior;
c) identifying a well-matched senior candidate for the senior living community based on senior attributes associated with the senior candidate and community attributes associated with the senior living community;
d) retrieving from the marketing plan dataset the marketing record linking the senior persona of the well-matched senior candidate with a care type, a marketing plan and a marketing budget;
e) calculating the price for the well-matched senior candidate and the senior living community based on the marketing plan for the senior persona of the well-matched senior, the marketing budget for the marketing plan, the community margin for the senior living community, the community cost for the senior living community, and the life expectancy for the well-matched senior candidate;
f) recording in a community info dataset a community record associating the senior living community with the well-matched senior candidate, the selected marketing plan, the marketing budget and the optimal price; and
g) transmitting at least a portion of the community record to the computing device controlled by the senior living community operator via the first data communications link.

9. The method of claim 6, further comprising automatically acquiring updated market condition data from the market conditions database on a periodic basis.

10. The method of claim 9, further comprising filtering and formatting the market condition data before storing the market condition data in the memory.
